(19) United States
(12) Patent Application Publication
(10) Pub. No.: US 2024/0359081 A1
(43) Pub. Date: Oct. 31, 2024

(54) INFORMATION PROCESSING APPARATUS, METHOD, AND NON-TRANSITORY COMPUTER-READABLE PROGRAM

(71) Applicant: SHOSABI INC., Tokyo (JP)

(72) Inventors: Sachiko YAMAGISHI, Tokyo (JP); Kenjiro KOBAYASHI, Tokyo (JP)

(73) Assignee: SHOSABI INC., Tokyo (JP)

(21) Appl. No.: 18/768,539

(22) Filed: Jul. 10, 2024

Related U.S. Application Data
(63) Continuation of application No. PCT/JP2022/044582, filed on Dec. 2, 2022.

(30) Foreign Application Priority Data
Jan. 24, 2022 (JP) 2022-008504

(51) Int. Cl. A63B 71/06; A63B 24/00; G06T 7/13; G06T 7/55; G06T 7/73; G06V 40/10

(52) U.S. Cl. CPC A63B 71/0622; A63B 24/0062; G06T 7/13; G06T 7/55; G06T 7/74; A63B 2071/063; A63B 2220/05; A63B 2220/807; A63B 2230/62; G06T 2207/10028; G06T 2207/30196; G06V 40/10

(57) ABSTRACT

An information processing apparatus comprising processing circuitry is provided. The processing circuitry is configured to perform: acquiring an input image captured by one or more image capturing apparatuses installed at a forward side or a rearward side of a reference point; determining a first contour of a target located in a vicinity of the reference point viewed from a first viewpoint based on a first partial image of the input image in which the target is captured and determining a second contour of the target viewed from a second viewpoint different from the first viewpoint based on a second partial image of the input image in which a mirror image of the target reflected by a lateral mirror installed at a side of the reference point is captured; and presenting information based on at least one of the first contour or the second contour.

### CROSS-REFERENCE TO RELATED APPLICATIONS

This application is based upon and claims the benefit of priority from the prior Japanese Patent Application No. 2022-008504, filed Jan. 24, 2022 and from PCT/JP2022/44582, filed Dec. 2, 2022, the entire contents of which are incorporated herein by reference.

### FIELD

Embodiments described herein relate generally to an information processing apparatus, a method, and a non-transitory computer-readable program.

### BACKGROUND

Posture is important in exercise. For example, if a particular part of the body is not appropriately moving, an excessive load may be placed on a muscle or joint near the part, the performance may decrease, and the risk of injury may increase.

Conventional system discloses a technique of generating guidance information that allows a subject to be aware of whether the subject is exercising using exercise equipment in an ideal manner based on a captured image acquired by capturing an image of a detection area for detecting the subject exercising using the exercise equipment, and depth information on the subject in the detection area.

In the Conventional system, an RGB camera is used to capture an image of the front of the subject, and a depth sensor is used to detect the depth of the front of the subject. However, the technique cannot provide information about the side or back of the subject.

If another RGB camera or depth sensor (referred to simply as a “camera” hereinafter) is installed at a side or the rear of the subject, information about the side or back of the subject can be obtained. However, when building such a system in a limited space, such as the home of the subject or a fitness gym, it may be difficult to properly arrange the cameras because the required distances for image capturing cannot be kept.

An object of the present disclosure is to achieve analysis of a target in a plurality of directions in a limited space.

### BRIEF DESCRIPTION OF DRAWINGS

FIG. 1 shows a block diagram showing a configuration of an information processing system according to an embodiment.

FIG. 2 shows a diagram for illustrating an installation environment of an image capturing apparatus in this embodiment.

FIG. 3 shows a diagram for illustrating an aspect of this embodiment.

FIG. 4 shows a diagram showing a subject viewed from the image capturing apparatus in this embodiment.

FIG. 5 shows a flowchart of information processing in this embodiment.

FIG. 6 shows a diagram showing an example of partial images included in an input image.

FIG. 7 shows a flowchart of a specific example of Step S111 in FIG. 5.

FIG. 8 shows a diagram showing an example of bones estimated from the partial images.

FIG. 9 shows a diagram showing an example of part recognition results.

FIG. 10 shows a diagram showing an example of contour extraction results.

FIG. 11 shows a diagram showing an example of posture evaluation results.

FIG. 12 shows a diagram showing an example of partial images included in an input image (RGB image).

FIG. 13 shows a flowchart of a variation of Step S111 in FIG. 5.

FIG. 14 shows a diagram showing bones estimated from the partial images.

FIG. 15 shows a diagram showing silhouettes of the partial images.

### DETAILED DESCRIPTION

In general, according to an embodiment, an information processing apparatus comprising processing circuitry is provided. The processing circuitry acquires an input image captured by one or more image capturing apparatuses installed at a forward side or a rearward side of a reference point; determines a first contour of a target located in a vicinity of the reference point viewed from a first viewpoint based on a first partial image of the input image in which the target is captured and determines a second contour of the target viewed from a second viewpoint different from the first viewpoint based on a second partial image of the input image in which a mirror image of the target reflected by a lateral mirror installed at a side of the reference point is captured; and presents information based on at least one of the first contour or the second contour.

In the following, an embodiment of the present invention will be described in detail with reference to the drawings. In the drawings for illustrating the embodiment, like components are denoted by reference like numerals, and redundant descriptions thereof will be omitted.

### (1) Configuration of Information Processing System

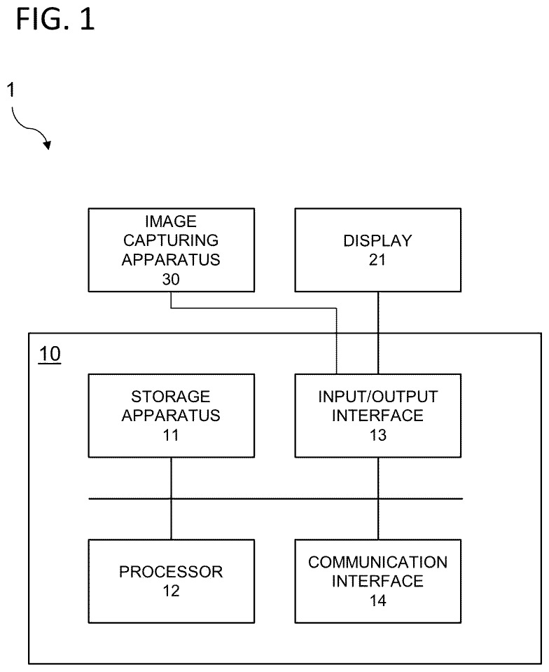

A configuration of an information processing system will be described. FIG. 1 is a block diagram showing a configuration of an information processing system according to the embodiment.

As shown in FIG. 1, an information processing system 1 includes an information processing apparatus 10, a display 21 and an image capturing apparatus 30.

The information processing apparatus 10 is a computer (such as a smartphone, a tablet terminal or a personal computer). The information processing apparatus 10 acquires an image captured by the image capturing apparatus 30 and performs processing on the image. The information processing apparatus 10 displays an image on the display 21 to present information to a user.

The display 21 is configured to display an image (a static image or a moving image). The display 21 is a liquid crystal display or an organic EL display, for example. There may be one or more displays 21.

The image capturing apparatus 30 in this embodiment includes a depth sensor, for example, and generates point cloud data by performing sensing. In the following description, image capturing includes sensing with the depth sensor, and image includes point cloud data. The image capturing apparatus 30 transmits an image (point cloud data) to the information processing apparatus 10.

### (1-1) Configuration of Information Processing Apparatus

A configuration of the information processing apparatus will be described.

As shown in FIG. 1, the information processing apparatus 10 includes a storage apparatus 11, a processor (processing circuitry) 12, an input/output interface 13 and a communication interface 14. The information processing apparatus 10 is connected to the display 21 and the image capturing apparatus 30.

The storage apparatus 11 is configured to store a program and data. The storage apparatus 11 is a combination of a read only memory (ROM), a random access memory (RAM) and a storage (such as a flash memory or a hard disk), for example.

For example, the program includes the following programs:

a program of an operating system (OS); and

a program of an application that executes information processing.

For example, the data includes the following data:

a database that is referred to in information processing; and

data acquired by executing information processing (that is, a result of execution of information processing).

The processor 12 is a computer that activates a program stored in the storage apparatus 11 to implement a function of the information processing apparatus 10. For example, the processor 12 is at least one of the followings:

a central processing unit (CPU);

a graphic processing unit (GPU);

an application specific integrated circuit (ASIC); and

a field programmable Gate array (FPGA).

The input/output interface 13 is configured to acquire information (such as an image or a user instruction) from an input device connected to the information processing apparatus 10 and output information (such as an image) to an output device connected to the information processing apparatus 10.

The input device is the image capturing apparatus 30, a keyboard, a pointing device, a touch panel or a combination thereof, for example.

The output device is the display 21, a speaker or a combination thereof, for example.

The communication interface 14 is configured to control communication between the information processing apparatus 10 and an external apparatus (such as a server (not shown)).

### (2) Aspect of Embodiment

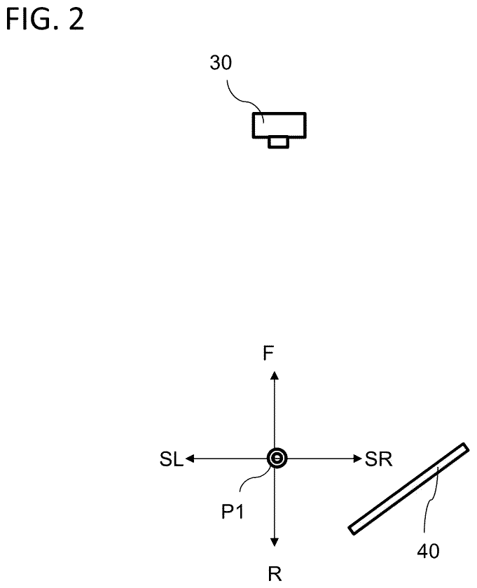

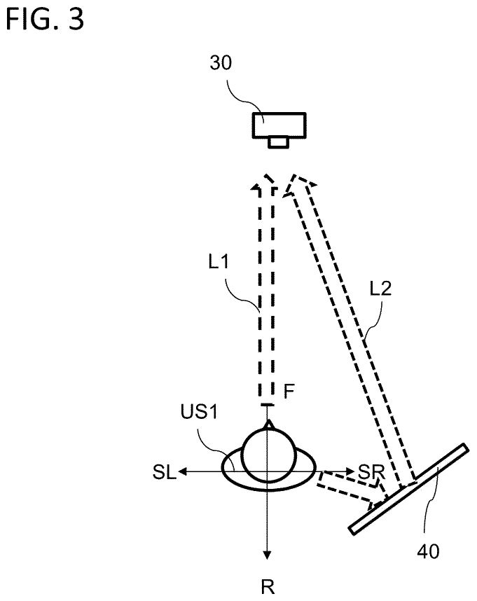

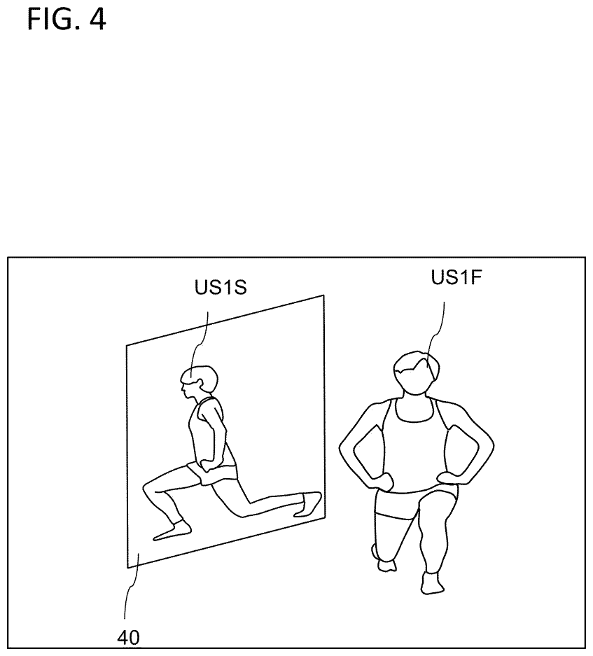

An aspect of this embodiment will be described. FIG. 2 is a diagram for illustrating an installation environment of the image capturing apparatus in this embodiment. FIG. 3 is a diagram for illustrating an aspect of this embodiment. FIG. 4 is a diagram showing a subject viewed from the image capturing apparatus in this embodiment.

As shown in FIG. 2, the image capturing apparatus 30 is placed at the forward (F) side of a reference point P1 in such a manner that the image capturing apparatus 30 can capture an image in the direction of the reference point P1. The reference point P1 is set at the center of an area in which the user is exercising, for example.

A lateral mirror 40 is installed at the right (SR) side of the reference point P1. The position and orientation of the lateral mirror 40 are determined so that the image capturing apparatus 30 can capture a mirror image of the user located near the reference point P1. The position of the lateral mirror 30 may be displaced more rearward (R) on the right side. Alternatively, the position of the lateral mirror 40 may be at the left (SL) side of the reference point P1 and may be displaced more rearward on the left side.

As shown in FIG. 3, in such an installation environment, it is assumed that a user US1 (an example of a “target”) exercises with the front of the body facing in the forward (F) direction of the reference point P1. Then, as shown in FIG. 4, the image capturing apparatus 30 can capture images of the user US1 viewed from different viewpoints (specifically, a front view US1F and a right-side view US1S) at the same time. Specifically, the image capturing apparatus 30 forms images from both light L1 that directly reaches the image capturing apparatus 30 from the front of the user US1 and light L2 that reaches the image capturing apparatus 30 from the right side of the user US1 after being reflected by the lateral mirror 40. In this way, the image capturing apparatus 30 generates an input image including a first partial image (frontal image) showing the front view of the user US1 and a second partial image (sagittal image) showing a mirror image of the right side of the user US1 reflected by the lateral mirror 40.

The information processing apparatus 10 acquires the input image from the image capturing apparatus 30, determines a contour (an example of a “first contour”) of the user US1 viewed from a viewpoint (an example of a “first viewpoint”) located at the forward (F) side of the reference point based on the first partial image, and determines a contour (an example of a “second contour”) of the user US1 viewed from a viewpoint (an example of a “second viewpoint”) located in the right (SR) direction of the reference point based on the second partial image. The information processing apparatus 10 presents, to the user US1, information based on at least one of the determined contours.

The distance from the reference point P1 to the lateral mirror 40 is defined to be equal to or greater than a minimum distance d1 required to capture the mirror image of the entire right (SR) side of the user US1. If another image capturing apparatus is installed at the right side of the reference point P1 instead of the lateral mirror 40, an initial distance d2 required for the image capturing apparatus to capture an image of the entire right side of the user US1 is greater than the distance d1. In other words, since the lateral mirror 40, rather than another image capturing apparatus, is installed at the right side of the reference point P1, and the image capturing apparatus 30 installed at the forward (F) side of the reference point P1 captures the mirror image, the space occupied by the information processing system 1 can be reduced in the lateral direction. Specifically, the image capturing apparatus 30 and the lateral mirror 40 can be installed in such a manner that the distance from the lateral mirror 40 to the reference point P1 is smaller than the distance from the image capturing apparatus 30 to the reference point P1. This allows a plurality of information processing systems 1 to be placed with high density in the lateral direction in a fitness gym.

With such an information processing system 1 according to this embodiment, the user US1 can be analyzed in a plurality of directions in a limited space, and information based on the analysis result can be fed back to the user US1. As such, the user US1 is prompted to improve posture.

### (3) Information Processing

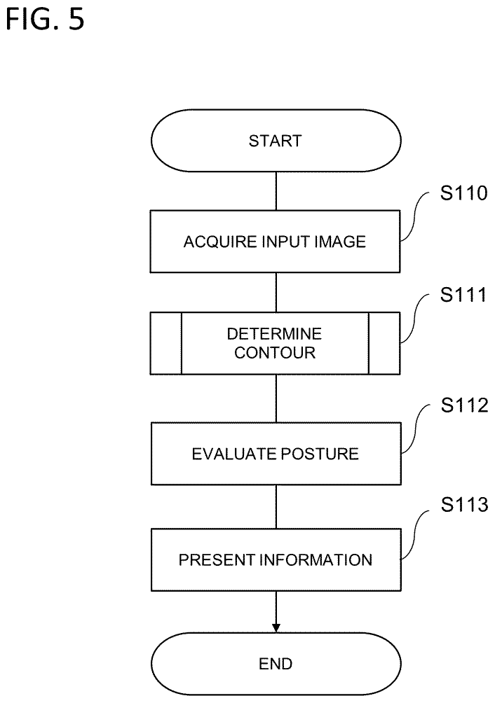

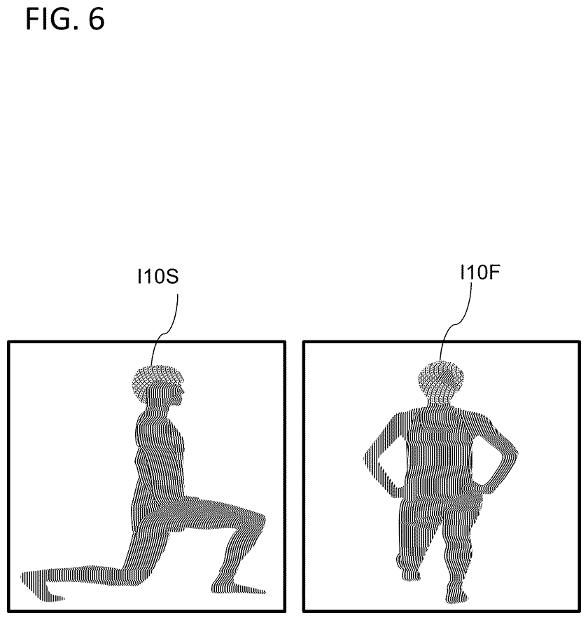

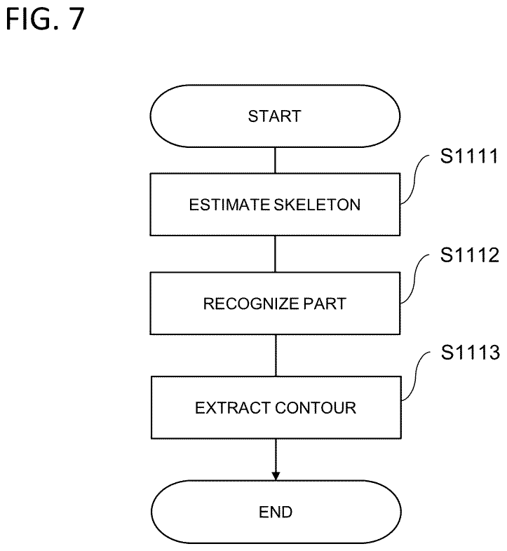

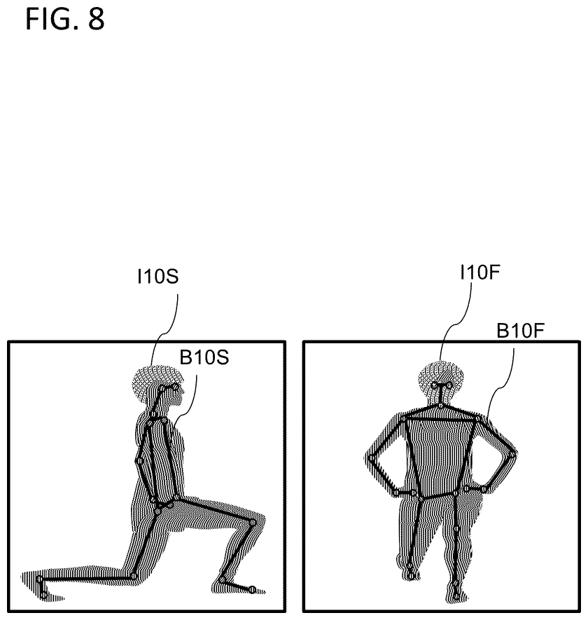

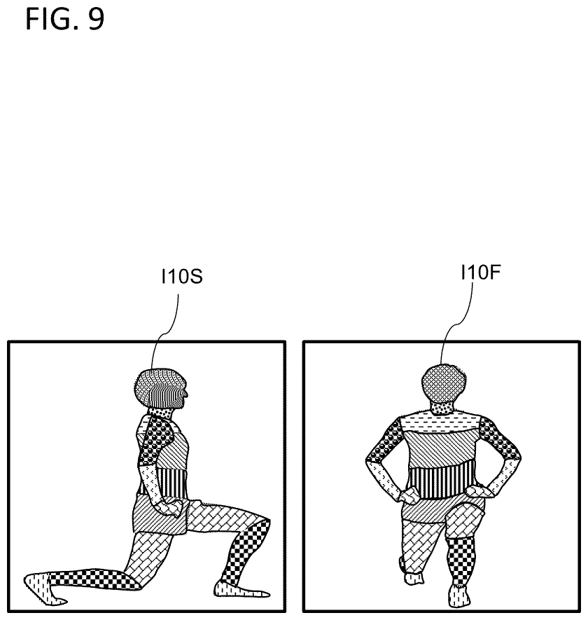

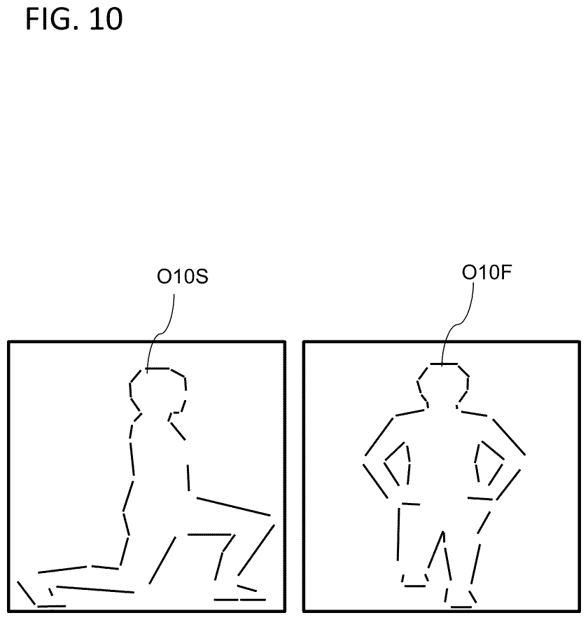

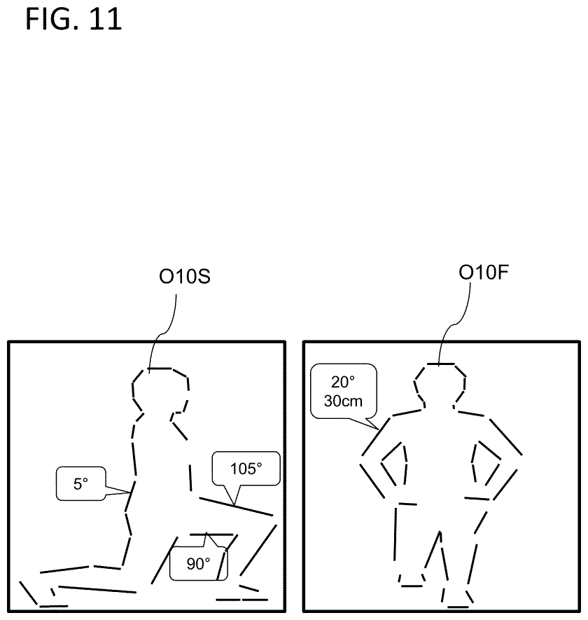

Information processing in this embodiment will be described. FIG. 5 is a flowchart of information processing in this embodiment. FIG. 6 is a diagram showing an example of the partial images included in the input image. FIG. 7 is a flowchart of a specific example of Step S111 in FIG. 5. FIG. 8 is a diagram showing an example of bones estimated from the partial images. FIG. 9 is a diagram showing an example of part recognition results. FIG. 10 is a diagram showing an example of contour extraction results. FIG. 11 is a diagram showing an example of posture evaluation results.

The information processing in FIG. 5 may be started in response to an operation by the user on the input device of the information processing apparatus 10 or may be automatically started on the condition that the user is detected in a vicinity of the reference point, for example. The vicinity of the reference point refers to a group of positions where the image capturing apparatus 30 can capture an image of the entire body of the user when the user is present at any of the positions.

As shown in FIG. 5, the information processing apparatus 10 performs acquisition of an input image (S110).

Specifically, the information processing apparatus 10 acquires an input image from the image capturing apparatus 30. As shown in FIG. 6, the input image includes a first partial image I10F in which the user located in the vicinity of the reference point is captured, and a second partial image I10S in which a mirror image of the user reflected by the lateral mirror 40 is captured. Although color coding is not used in the example in FIG. 6, points of point cloud data can be color-coded according to the depth.

After Step S110, the information processing apparatus 10 performs contour determination (S111).

Specifically, the information processing apparatus 10 determines a contour of the user based on the first partial image and the second partial image included in the input image acquired in Step S110. The information processing apparatus 10 determines a first contour of the user viewed from a first viewpoint based on the first partial image. The first viewpoint depends on the direction in which the image capturing apparatus 30 is installed, and is located at the forward (F) side of the reference point in this embodiment. The information processing apparatus 10 determines a second contour of the user viewed from a second viewpoint different from the first viewpoint based on the second partial image. The second viewpoint depends on the direction in which the lateral mirror 40 is installed, and is located at the right (SR) side of the reference point in this embodiment.

In the following, the contour determination (S111) in this embodiment will be described in detail.

As shown in FIG. 7, the information processing apparatus 10 performs skeleton estimation (S1111).

Specifically, the information processing apparatus 10 performs skeleton estimation processing on the first partial image and the second partial image included in the input image acquired in Step S110. In this processing, as shown in FIG. 8, the information processing apparatus 10 obtains bones B10F in the first partial image I10F and bones B10S in the second partial image I10S.

After Step S1111, the information processing apparatus 10 performs part recognition (S1112).

Specifically, the information processing apparatus 10 refers to the bones estimated in Step S1111 to recognize a correspondence between the point clouds forming the first partial image and the second partial image and parts of the body of the user. In this processing, as shown in FIG. 9, the first partial image I10F and the second partial image I10S are divided into point cloud regions corresponding to parts of the body of the user.

After Step S1112, the information processing apparatus 10 performs contour extraction (S1113).

Specifically, the information processing apparatus 10 extracts contours of parts of the body of the user from an envelope (that is, an envelope line or an envelope surface) of the body of the user based on the recognition result in Step S1112. As shown in FIG. 10, the contour is a straight line (line segment). Extraction of a plurality of contours on a part basis allows a complicated postural distortion, which is difficult to evaluate based only on the skeleton estimation result, to be quantitatively evaluated or visualized in an understandable manner. The information processing apparatus 10 ends the contour determination (S111) with Step S1113.

As shown in FIG. 5, after Step S111, the information processing apparatus 10 performs posture evaluation (S112).

Specifically, the information processing apparatus 10 evaluates the postures of parts of the user based on the contours determined in Step S111. The parts can include at least one of head, neck, shoulder, chest, belly, back, waist, hip, brachium, forearm, hand, thigh, crus or foot, for example.

In a first example, the information processing apparatus 10 measures the angle and length of a contour (line) of a part of the body of the user.

In a second example, the information processing apparatus 10 evaluates the posture of a part of the body of the user based on a comparison between the contour (line) of the part and a corresponding reference contour line. The comparison can be made on the angle, the length or a combination thereof. The angle of the reference contour line may be determined according to the kind of exercise performed by the user. The length of the reference contour line may be determined accordingly based on a result of measurement of the body of the user or a result of classification of the physique of the user. Specific examples of the evaluation are as follows.

The information processing apparatus 10 evaluates the degree of distortion (forward tilt or backward tilt) of the pelvis of the user or the degree of bending of the back or waist of the user based on a comparison between the contour line of the back of the user and a reference contour line corresponding to the back.

The information processing apparatus 10 evaluates the orientation of the toe of the user based on a comparison between the contour line of the toe of the user and a reference contour line corresponding to the toe.

The information processing apparatus 10 detects a motion of the user shrugging the shoulders based on a comparison between the contour line of the shoulders of the user and a reference contour line corresponding to the shoulders.

The information processing apparatus 10 evaluates the degree of raising of the chin of the user or the lateral inclination of the face of the user based on a comparison between the contour line of the face of the user and a reference contour line corresponding to the face.

The kind of exercise may be specified by the user, a trainer who instructs the user how to exercise or an administrator of the information processing system 1, or may be recognized based on the motion of the user. The kind of exercise may be at least one of the following basic motions:

push;

pull;

plank;

rotate;

hinge;

lunge; and

squat.

After Step S112, the information processing apparatus 10 performs information presentation (S113).

Specifically, the information processing apparatus 10 presents various kinds of information to the user through the output device. The information processing apparatus 10 ends the information processing in this embodiment with Step S113.

The information processing apparatus 10 may presents at least one of the following:

an image captured by the image capturing apparatus 30 or a part of the image;

information about the three-dimensional shape of the body of the user estimated based on the first partial image and the second partial image;

information about the result of the contour determination in Step S111;

information about the result of the posture evaluation in Step S112; and

information about advice for the user (such as advice to improve the posture of a particular part of the body of the user) based on the result of the posture evaluation in Step S112.

The information processing apparatus 10 may present audio including the information described above to the user through a speaker or may present an image including the information described above to the user on the display 21. The display 21 for presenting the information described above may be installed in the forward (F) direction of the reference point. In addition, a half mirror may be installed between the display 21 and the reference point. In this case, the user can see the front view of the user reflected in the half mirror at normal times and can see what is displayed on the display 21 through the half mirror when information is presented.

### (4) Brief Summary

As described above, the information processing apparatus 10 in this embodiment determines the first contour of the body of the user based on the first partial image of the input image captured by the image capturing apparatus 30, in which the user located in the vicinity of the reference point is captured, and determines the second contour of the body of the user based on the second partial image of the input image, in which the mirror image of the user reflected by the lateral mirror 40 is captured. The information processing apparatus 10 presents, to the user, information based on at least one of the first contour or the second contour. As such, the contour of the body of the user can be analyzed in two directions, and at the same time the space required at the right side or left side of the user can be reduced.

The information processing apparatus 10 in this embodiment may evaluate the posture of a part of the body of the user exercising in the vicinity of the reference point based on at least one of the first contour or the second contour, and present information based on the evaluation result to the user. As such, the user can recognize whether the user is exercising in an appropriate posture. The information based on the evaluation result may be advice about the posture of a part of the body of the user. As such, the user can be notified of how to improve the posture. The information processing apparatus 10 may present audio including such advice to the user. As such, the user can be notified of how to improve the posture even when the user is not paying attention to the display 21.

In this embodiment, a half mirror may be installed in front of the reference point, and the display 21 may be installed behind the half mirror with respect to the reference point. In this case, the information processing apparatus 10 in this embodiment may display information on the display 21. As such, the user can see the front view of the user reflected in the half mirror at normal times and can see what is displayed on the display 21 through the half mirror when information is presented.

The information processing apparatus 10 in this embodiment may evaluate the angle of the contour line of a part of the body of the user based on at least one of the first contour or the second contour. As such, the posture of the part of the body of the user can be quantitatively evaluated.

The information processing apparatus 10 in this embodiment may determine a contour line of a part of the body of the user based on at least one of the first contour or the second contour and evaluate the posture of the part based on a comparison between the contour line and a reference contour line. As such, the posture of the part of the body of the user can be evaluated based on a deviation from an ideal posture. The information processing apparatus 10 may determine a contour line of the back of the user based on at least one of the first contour or the second contour and evaluate the distortion of the pelvis of the user or the bending of the back or waist of the user based on a comparison between the contour line and a reference contour line. As such, the distortion of the pelvis or the bending of the back or waist, which are difficult to evaluate based only on the skeleton estimation result, can be quantitatively evaluated. The information processing apparatus 10 may determine a contour line of the toe of the user based on at least one of the first contour or the second contour and evaluate the orientation of the toe of the user based on a comparison between the contour line and a reference contour line. As such, the orientation of the toe, which is difficult to evaluate based only on the skeleton estimation result, can be quantitatively evaluated.

In this embodiment, the image capturing apparatus 30 and the lateral mirror 40 may be installed in such a manner that the distance from the lateral mirror 40 to the reference point is smaller than the distance from the image capturing apparatus 30 to the reference point. As such, the space required at the right side or left side of the user can be reduced compared with the space required in front of the user.

### (5) Variations

Variations of the embodiment will be described.

### (5-1) Variation 1

A variation 1 will be described. The variation 1 is an example in which an image capturing apparatus 30 including an RGB camera is used.

### (5-1-1) Information Processing

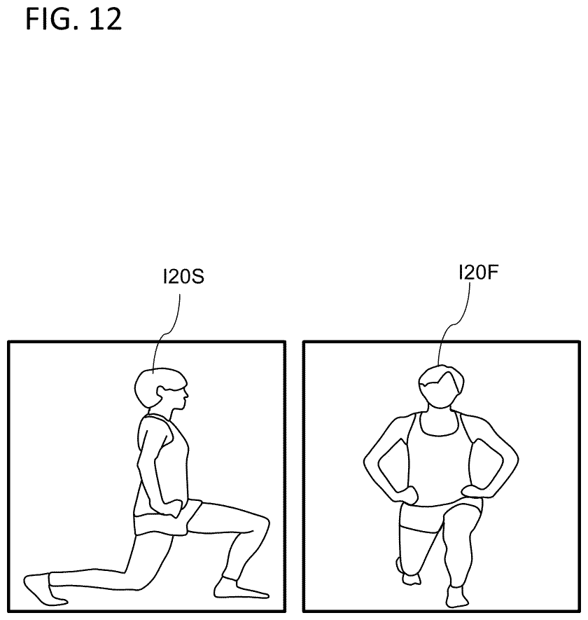

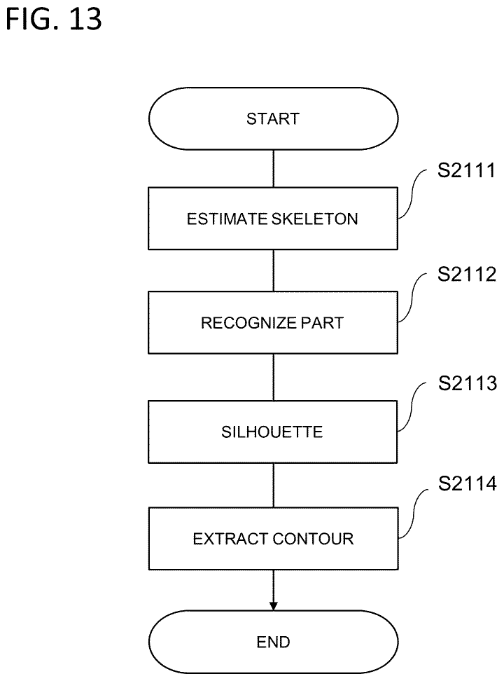

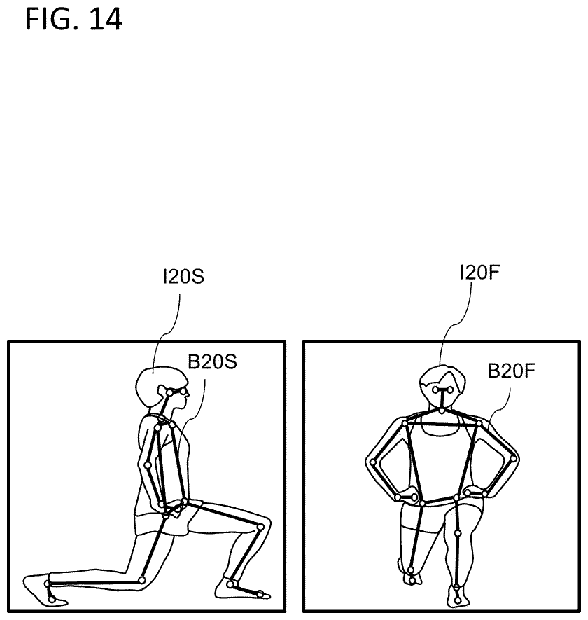

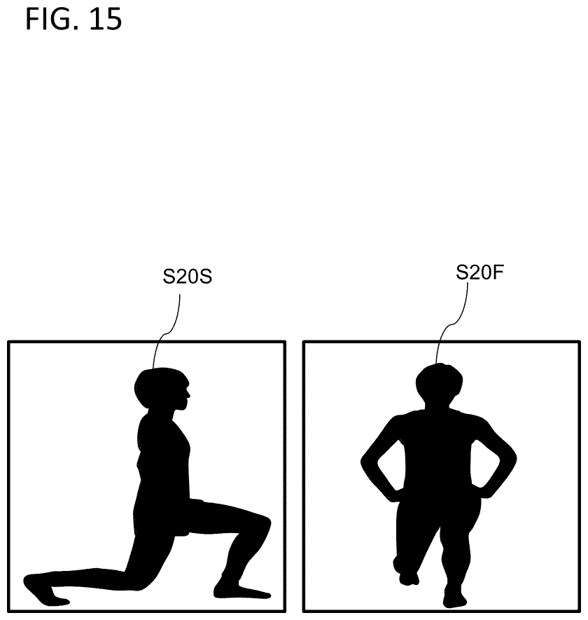

Information processing in the variation 1 will be described. FIG. 12 is a diagram showing an example of partial images included in an input image (RGB image). FIG. 13 is a flowchart of a variation of Step S111 in FIG. 5. FIG. 14 is a diagram showing bones estimated from the partial images. FIG. 15 is a diagram showing silhouettes of the partial images.

As in the process in FIG. 5, the information processing apparatus 10 performs acquisition of an input image (S110).

Specifically, the information processing apparatus 10 acquires an input image from the image capturing apparatus 30. As shown in FIG. 12, the input image includes a first partial image I20F in which the user located in the vicinity of the reference point is captured, and a second partial image I20S in which a mirror image of the user reflected by the lateral mirror 40 is captured.

After Step S110, as in the process in FIG. 5, the information processing apparatus 10 contour performs determination (S111).

Specifically, the information processing apparatus 10 determines a contour of the user based on the first partial image and the second partial image included in the input image acquired in Step S110. The information processing apparatus 10 determines a first contour of the user viewed from a first viewpoint based on the first partial image. The information processing apparatus 10 determines a second contour of the user viewed from a second viewpoint different from the first viewpoint based on the second partial image.

In the following, the contour determination (S111) in the variation 1 will be described in detail.

As shown in FIG. 13, the information processing apparatus 10 performs skeleton estimation (S2111).

Specifically, the information processing apparatus 10 performs skeleton estimation processing on the first partial image and the second partial image included in the input image acquired in Step S110. As such, as shown in FIG. 14, the information processing apparatus 10 obtains bones B20F in the first partial image I20F and bones B20S in the second partial image I20S.

After Step S2111, the information processing apparatus 10 performs part recognition (S2112).

Specifically, the information processing apparatus 10 performs three-dimensional alignment of the first partial image and the second partial image. As described above, the first partial image represents the appearance of the user viewed from the first viewpoint, and the second partial image represents a mirror image of the user viewed from the second viewpoint. Therefore, if the positional relationship between the image capturing apparatus 30 and the lateral mirror 40, the orientation of the image capturing apparatus 30 and the orientation of the lateral mirror 40 are known, for corresponding points in the first partial image and the second partial image, the information processing apparatus 10 can calculate the distance (that is, depth) between the corresponding point and the first viewpoint or second viewpoint. That is, the information processing apparatus 10 can acquire information about the three-dimensional shape of the body of the user at corresponding points in the first partial image and the second partial image.

The information processing apparatus 10 then refers to the bones estimated in Step S2111 to recognize a correspondence between pixels forming the first partial image and the second partial image and parts of the body of the user. In this processing, the first partial image and the second partial image are divided into pixel regions corresponding to parts of the body of the user. Furthermore, for pixels at corresponding points in the first partial image and the second partial image, the information processing apparatus 10 may refer to the depths of the pixels to recognize a correspondence between the pixels and a part of the body of the user.

After Step S2112, the information processing apparatus 10 performs silhouetting (S2113).

Specifically, the information processing apparatus 10 performs silhouette processing on the first partial image and the second partial image included in the input image acquired in Step S110. In this processing, as shown in FIG. 15, the information processing apparatus 10 obtains a silhouette image S20F of the first partial image I20F and a silhouette image S20S of the second partial image I20S.

After Step S2113, the information processing apparatus 10 performs contour extraction (S2114).

Specifically, the information processing apparatus 10 extracts contours of parts of the body of the user from an envelope line in the silhouette images generated in Step S2113 based on the recognition result in Step S2112. Furthermore, for pixels at corresponding points in the first partial image and the second partial image, the information processing apparatus 10 may refer to the depths of the pixels to estimate an envelope (that is, an envelope line or an envelope surface) of the part and extract a contour from the envelope. As in the case of the embodiment, the contour is a straight line (line segment). By extracting a plurality of contours on a part basis, a postural distortion that is difficult to evaluate based only on the skeleton estimation result can be quantitatively evaluated or visualized in an understandable manner. The information processing apparatus 10 ends the contour determination (S111) with Step S2114.

As in the process in FIG. 5, after Step S111, the information processing apparatus 10 performs posture evaluation (S112) and information presentation (S113). The information processing apparatus 10 ends the information processing in the variation 1 with Step S113.

### (5-1-2) Brief Summary

As described above, the information processing apparatus 10 in the variation 1 uses the image capturing apparatus 30 including an RGB camera to determine a contour of a part of the body of the user and presents information based on the contour. As such, the cost of the image capturing apparatus 30 can be reduced compared with the embodiment.

The information processing apparatus 10 in the variation 1 may calculate the depths of corresponding points in the first partial image and the second partial image based on the positional relationship between the image capturing apparatus 30 and the lateral mirror 40 and the orientations of the image capturing apparatus 30 and the lateral mirror 40 and determine a contour or evaluate the posture of a part of the body of the user based on the depths. As such, contour determination or posture estimation based on information about the three-dimensional shape of some parts of the body of the user can be performed without using a depth sensor.

### (5-2) Variation 2

A variation 2 will be described. The variation 2 is an example in which information analysis and presentation are not performed in a three-dimensional domain in the embodiment or the variation 1.

In a first example, the contour extraction (S1113) in the embodiment is modified as follows. Specifically, the information processing apparatus 10 in the variation 2 extracts contours of parts of the body of the user from an (two-dimensional) envelope line of the body of the user based on the recognition result in Step S1112. The envelope line is a curve on an envelope surface of the body of the user that intersects with a plane corresponding to the first partial image and a plane corresponding to the second partial image. The plane corresponding to the first partial image is defined as a plane that is perpendicular to the front and rear (F-R) direction, for example, and the plane corresponding to the second partial image is defined as a plane that is parallel to the mirror surface of the lateral mirror 40, for example.

The information presentation (S113) in the embodiment is modified so that information about the three-dimensional shape of the body of the user estimated based on the first partial image and the second partial image is not presented.

In a second example, the part recognition (S2112) in the variation 1 is modified so that three-dimensional alignment of the first partial image and the second partial image is not performed (that is, the depths of pixels are not calculated) and the recognition of a correspondence between pixels the depths of which are referred to and a part of the body of the user is not performed. Furthermore, the contour extraction (S2114) in the variation 1 is modified so that the estimation of an envelope of a part for which the depths of pixels are referred to and the extraction of a contour from the envelope are not performed.

### (5-3) Variation 3

A variation 3 will be described. The variation 3 is an example in which various kinds of useful information are presented to a user or a person who provides a service regarding exercise to the user (such as a personal trainer, a staff member of a training facility or an intermediary between the user and the personal trainer or training facility, referred to as a “service provider” hereinafter) based on a posture evaluation result.

In a first example, the information processing apparatus 10 can determine a critical part of the user based on the posture evaluation result. The critical part refers to a part of the body of the user in which appropriate movement of the body is impaired because the part is relatively poor in muscle strength, endurance, flexibility, balance ability or a combination thereof, and which needs to be intensively built up by the user. The information processing apparatus 10 may present information about a critical part to the user or the service provider. If the information about a critical part is presented to the service provider, the service provider can determine the service for the user by considering the critical part of the user. Furthermore, based on the information about a critical part of the user, the information processing apparatus 10 may introduce, to the user, a trainer (a personal trainer or a trainer at a training facility) who is good at training the critical part. Alternatively, based on the information about a critical part of the user, the information processing apparatus 10 may inform the user of a kind of exercise, a training machine or a training facility that is appropriate for training of the critical part. Furthermore, based on the information about a critical part of the user, the information processing apparatus 10 may automatically create a training menu including a plurality of kinds of exercise for the user and present the training menu to the user or the service provider. The kinds of exercise included in the training menu may be selected based on the training machines that can be provided by the service provider, for example. Information about a part the training of which the trainer is good at can be managed in a database (not shown). Similarly, information about a kind of exercise, a training machine or a training facility that is appropriate for each part can be managed in the database (not shown).

In a second example, the information processing apparatus 10 may determine a bad-condition part of the user based on the posture evaluation result. The bad-condition part refers to a part of the body of the user that has poorer movement than usual. The information processing apparatus 10 may refer to information about contours collected in the past for the user, in order to determine a bad-condition part of the user. The information processing apparatus 10 may present information about a bad-condition part to the user or the service provider. If the information about a bad-condition part is presented to the service provider, the service provider can determine the service for the user by considering the bad-condition part of the user. Furthermore, based on the information about a bad-condition part of the user, the information processing apparatus 10 may introduce, to the user, a trainer (a personal trainer or a trainer at a training facility) who is good at conditioning or training the bad-condition part. Alternatively, based on the information about a bad-condition part of the user, the information processing apparatus 10 may inform the user of a kind of exercise, a training machine or a training facility that is appropriate for conditioning or training of the bad-condition part. Furthermore, based on the information about a bad-condition part of the user, the information processing apparatus 10 may automatically create a training menu including a plurality of kinds of exercise for the user and present the training menu to the user or the service provider. The kinds of exercise included in the training menu may be selected based on the training machines that can be provided by the service provider, for example. Information about a part the training of which the trainer is good at can be managed in a database (not shown). Similarly, information about a kind of exercise, a training machine or a training facility that is appropriate for training or conditioning of each part can be managed in the database (not shown).

### (6) Other Variations

The storage apparatus 11 may be connected to the information processing apparatus 10 via a network NW. The display 21 may be integrated with or external to the information processing apparatus 10.

In the embodiment, an example in which the image capturing apparatus 30 is installed at the forward (F) side of the reference point has been shown.

However, the image capturing apparatus 30 may be installed at the rearward (R) side of the reference point. In this case, a front mirror may be installed in addition to or instead of the lateral mirror 40. The front mirror is installed at the forward side of the reference point. The front mirror may be a half mirror, and in this case, the combination of the front mirror and the display 21 allows information (such as an image of the back of the user) displayed on the display 21 to be presented to the user in the vicinity of the reference point through the front mirror. Displaying the image of the back of the user allows the user to observe the back of the user, which the user has little opportunity to see.

Alternatively, the image capturing apparatus 30 may be installed at the left (SL) side or right (SR) side of the reference point. In this case, the lateral mirror 40 is installed on the opposite side of the reference point to the image capturing apparatus 30. Alternatively, a front mirror or a back mirror may be installed instead of the lateral mirror 40. The front mirror is installed at the forward side of the reference point, and the back mirror is installed at the rearward side of the reference point. The front mirror may be a half mirror, and in this case, the combination of the front mirror and the display 21 allows information (such as an image of the back of the user) displayed on the display 21 to be presented to the user in the vicinity of the reference point through the front mirror.

In the embodiment, an example has been shown in which the lateral mirror 40 is installed at the right (SR) side or left (SL) side of the reference point. However, in addition to or instead of the lateral mirror 40, a front mirror may be installed at the forward (F) side of the reference point. In this case, if the image capturing apparatus 30 is installed at the rearward (R) side, right side or left side of the reference point, a mirror image of the front of the user can be captured. Similarly, in addition to or instead of the lateral mirror 40, a back mirror may be installed at the rearward (R) side of the reference point. In this case, if the image capturing apparatus 30 is installed at the forward side, right side or left side of the reference point, a mirror image of the back of the user can be captured.

If the image capturing apparatus 30 and a mirror are installed in such a manner that the image capturing apparatus 30 and the mirror are positioned on the opposite sides of the reference point (for example, the image capturing apparatus 30 is installed at the forward (F) side of the reference point, and a back mirror is installed at the rearward (R) side of the reference point), the space occupied by the information processing system 1 can be linear (or narrow).

The information processing apparatus 10 may determine whether the second partial image includes the entire body of the user or not and prompt the user to move so that the entire body of the user is included in the second partial image. In particular, when the image capturing apparatus 30 and the mirror are positioned on the opposite sides of the reference point, there is a possibility that the capturing of the mirror image of the user is prevented by the body of the user. By disposing the image capturing apparatus 30 and the mirror so as to be opposed to each other and prompting the user to move as required, the space occupied by the information processing system 1 can be linear, and at the same time acquisition of an input image appropriate for analysis can be facilitated.

One of a plurality of image capturing apparatuses 30 (referred to as a “top image capturing apparatus 30T” hereinafter) may be installed at the upper side of the reference point (that is, the ceiling side). The top image capturing apparatus 30T captures an image in the downward direction (that is, in the direction toward the floor) and generates an input image including a third partial image (a traverse plane image) of the user viewed from above. In this case, the information processing apparatus 10 can determine a contour (an example of a “third contour”) of the user viewed from a viewpoint (an example of a “third viewpoint”) located above the reference point based on the third partial image and further perform posture evaluation or information presentation based on the contour. As such, the posture of a part that can be appropriately observed from above (such as the orientation of the toe) can be evaluated with high precision, for example. Alternatively, instead of the top image capturing apparatus 30T, a top mirror may be installed at the upper side of the reference point, and one of a plurality of image capturing apparatuses 30 (referred to as a “top-mirror image capturing apparatus 30B” hereinafter) may be installed below the top mirror. The top-mirror image capturing apparatus 30B captures an image in the upward direction and generates an input image including a third partial image (a traverse plane image) in which a mirror image of the user reflected by the top mirror is captured. In this case, the information processing apparatus 10 can determine a contour (an example of the “third contour”) of the user viewed from a viewpoint (an example of the “third viewpoint”) located above the reference point based on the third partial image and further perform posture evaluation or information presentation based on the contour. As such, the posture of a part that can be appropriately observed from above (such as the orientation of the toe) can be evaluated with high precision, for example.

In the embodiment, examples in which one image capturing apparatus 30 is used have been shown. However, a plurality of image capturing apparatuses 30 may be used in combination.

In a first example, a first image capturing apparatus 30-1 includes a depth sensor, and a second image capturing apparatus 30-2 includes an RGB camera. For example, skeleton estimation may be performed based on an image captured by the second image capturing apparatus 30-2, and a contour may be extracted from an image captured by the first image capturing apparatus 30-1 based on the estimation result.

In a second example, a first image capturing apparatus 30-1 is installed at any of the following positions, and a second image capturing apparatus 30-2 is installed at any of the following positions that is different from the location of the first image capturing apparatus 30-1:

at the forward (F) side of the reference point;

at the rearward (R) side of the reference point;

at the left (SL) side of the reference point; and

at the right (SR) side of the reference point.

In a third example, both a first image capturing apparatus 30-1 and a second image capturing apparatus 30-2 are installed at the forward (F) side, the rearward (R) side, the left (SL) side or the right (SR) side of the reference point, the first image capturing apparatus 30-1 is adjusted to focus on the vicinity of the reference point, and the second image capturing apparatus 30-2 is adjusted to focus on the vicinity of a mirror. The information processing apparatus 10 extracts a first partial image from an input image captured by the first image capturing apparatus 30-1 and extracts a second partial image from an input image captured by the second image capturing apparatus 30-2. As such, the first partial image and the second partial image can be clearly acquired, and contour determination and posture evaluation can be performed with high precision.

In the embodiment, examples in which the posture of a part of the body of the user is evaluated have been shown. The posture evaluation result may be used as an operation input for an object in a virtual space. That is, based on the posture evaluation result of a part of the body of the user, the information processing apparatus 10 may control the posture of the corresponding part of an operation target (for example, an object such as an avatar) in a virtual space. As such, the user can use the body of the user to intuitively move the operation target in the virtual space.

When the user performs a kind of exercise (such as dead lift, bench press, running on a treadmill, yoga or pedaling on a stationary bike) including a repetition of unit movements, the information processing apparatus 10 may measure the time required by the user to complete one cycle of movements and present information (such as a caution about overwork) to the user when the required time exceeds a reference value. The required time may be measured based on an image, the skeleton or the contour of the user, for example. The reference value may be determined in advance or may be a minimum required time measured for the user (such as the required time for the first unit movement) multiplied by a predetermined ratio (such as 1.2).

The information processing apparatus 10 may determine the contour of exercise equipment (such as a barbell, a dumbbell or a kettlebell) around the user in addition to the contour of a part of the body of the user, and perform posture evaluation or information presentation based on the contour.

In a first example, when the user performs dead lifts, the information processing apparatus 10 may determine whether the bar is lowered to the positions of the crura of the user and present information (such as a caution about an inappropriate posture or advice to lower the bar to the positions of the crura) to the user when the bar is not lowered to the positions of the crura of the user.

In a second example, when the user performs bench presses, the information processing apparatus 10 may evaluate the angle of the contour of the bar and present information (such as a caution about an inappropriate posture or advice to keep the bar horizontal) to the user when it is detected that the bar is not horizontal.

In the variation 1, an example has been shown in which the input image (RGB image) is silhouetted and a contour is extracted from the silhouette image. However, even when the input image is point cloud data, the input image may be silhouetted, and a contour may be extracted from the silhouette image. As such, the boundary of a point cloud is clearly defined, and contour extraction is facilitated. In addition, the same algorithm can be applied regardless of the format of the input image (whether RGB image or point cloud data), modules can be shared.

When silhouetting an input image in a format (RGB image or point cloud data), the entire body of the user is not necessarily silhouetted. In other words, a part of the input image that corresponds to a particular part of the body of the user (referred to as a “to-be-silhouetted part” hereinafter) may be silhouetted to extract the contour. As such, even when a part overlaps with another part in the input image, the contour can be easily extracted. The information processing apparatus 10 may exclude a part of the input image that corresponds to a part other than the to-be-silhouetted part from contour extraction candidates, and extract a contour without silhouetting the part.

The to-be-silhouetted part may be a specific part fixedly determined, such as arm, or may be dynamically determined based on various parameters. In a first example, the to-be-silhouetted part may be determined based on depth information. For example, the information processing apparatus 10 may select, as a to-be-silhouetted part, a part located at the forward (F) side with respect to a reference depth (such as the depth of the head, the chest, the belly or the waist). In a second example, the to-be-silhouetted part may be determined based on the kind of exercise of the user. For example, for a kind of exercise that involves a large movement of hands or arms, the information processing apparatus 10 may select brachia, forearms or hands as to-be-silhouetted parts. For example, for a kind of exercise that involves a large movement of feet or legs, the information processing apparatus 10 may select thighs, crura or feet as to-be-silhouetted parts.

In the embodiment, examples have been described in which the user performs a kind of exercise the user wants to do or a kind of exercise specified by the trainer. However, the information processing system 1 may propose a kind of exercise to be performed by the user. For example, the information processing system 1 may acquire information about activities of the user from a wearable device worn by the user and determine a kind of exercise to be proposed to the user. Specifically, the information processing system 1 may propose a strengthening training for legs to a user who sits for a long time. Alternatively, the information processing system 1 may randomly determine a kind of exercise to be proposed to the user. Furthermore, the information processing system 1 may determine a kind of exercise to be proposed next based on the posture of the user performing the kind of exercise proposed.

Information collected from a user or presented to a user or a service provider in one information processing system 1 may be shared with another information processing system 1. As such, the user can accumulate information about the body of the user and receive a more personalized service based on the accumulated information, without continuously using the same information processing system 1.

In a first example, information may be shared among a plurality of information processing systems 1 installed in the same training facility. In a second example, information may be shared among a plurality of information processing systems 1 installed in different training facilities of the same affiliated group. In a third example, a plurality of information processing systems 1 installed at different places (such as the home of the user and training facilities of different affiliated groups) are connected to a common server (such as a cloud server) via a network, and information may be accumulated in the cloud server. In the third example, the information processing system 1 used by the user can temporarily collect or present information on the condition that user authentication succeeds, and the information can be transferred to the server after the use by the user.

In the embodiment, examples have been shown in which an image of the user (that is, a human being) located in the vicinity of the reference point is captured. However, the target of the image capturing is not limited to a human being and may be a variety of living organisms or objects.

Examples have been shown in which the information processing system according to the embodiment is implemented as a stand-alone computer. However, the information processing system according to the embodiment may be implemented as a client/server system or a peer-to-peer system. In this case, each step of the information processing can be implemented by any apparatus. Although examples in which the steps of the processes are executed in particular orders have been shown in the above description, the execution order of the steps is not limited to the examples in the above description unless there is a dependence relationship between steps.

### (8) Appendixes

Items described in the embodiment and variations will be listed below as appendixes.

### Appendix 1

An information processing apparatus (10) comprising processing circuitry (12) configured to:

acquire (S110) an input image captured by one or more image capturing apparatuses (30) installed at a forward side or a rearward side of a reference point;

determine (S111) a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a lateral mirror (40) installed at a side of the reference point is captured; and

present (S113) information based on at least one of the first contour or the second contour.

### Appendix 2

The information processing apparatus according to appendix 1, wherein the target is a user exercising in the vicinity of the reference point, and

the processing circuitry is configured to:

evaluate (S112) a posture of a part of a body of the user based on at least one of the first contour or the second contour, and

present, to the user, information based on an evaluation result of the posture of the part of the body of the user.

### Appendix 3

The information processing apparatus according to appendix 2, wherein the processing circuitry is configured to present, to the user, advice concerning the posture of the part of the body of the user.

### Appendix 4

The information processing apparatus according to appendix 3, wherein the processing circuitry is configured to present, to the user, audio including the advice concerning the posture of the part of the body of the user.

### Appendix 5

The information processing apparatus according to any one of appendixes 2 to 4, wherein a half mirror is installed in front of the reference point, and

the processing circuitry is configured to display the information on a display (21) installed behind the half mirror with respect to the reference point.

### Appendix 6

The information processing apparatus according to appendix 5, wherein at least one of the image capturing apparatuses is installed at the rearward side of the reference point, and

the processing circuitry is configured to display a back image of the user based on the first partial image on the display.

### Appendix 7

The information processing apparatus according to any one of appendixes 2 to 6, wherein the processing circuitry is configured to:

calculate depths of corresponding points in the first partial image and the second partial image based on a positional relationship between the image capturing apparatus and the lateral mirror and orientations of the image capturing apparatus and the lateral mirror, and

evaluate the posture of the part of the body of the user based on the depths.

### Appendix 8

The information processing apparatus according to any one of appendixes 2 to 7, wherein the processing circuitry is configured to evaluate an angle of a contour line of the part of the body of the user based on at least one of the first contour or the second contour.

### Appendix 9

The information processing apparatus according to any one of appendixes 2 to 8, wherein the processing circuitry is configured to:

determine a contour line of the part of the body of the user based on at least one of the first contour or the second contour, and

evaluate the posture of the part of the body based on a comparison between the contour line of the part of the body of the user and a reference contour line.

### Appendix 10

The information processing apparatus according to appendix 9, wherein the processing circuitry is configured to:

determine a contour line of a back of the user based on at least one of the first contour or the second contour, and

evaluate a distortion of a pelvis of the user or a bending of the back or a waist of the user based on a comparison between the contour line of the back of the user and a reference contour line.

### Appendix 11

The information processing apparatus according to appendix 9, wherein the processing circuitry is configured to:

determine a contour line of a toe of the user based on at least one of the first contour or the second contour, and

evaluate an orientation of the toe of the user based on a comparison between the contour line of the toe of the user and a reference contour line.

### Appendix 12

The information processing apparatus according to any one of appendixes 2 to 11, wherein the processing circuitry is configured to control a posture of a corresponding part of an operation target in a virtual space in response to the evaluation result of the posture of the part of the body of the user.

### Appendix 13

The information processing apparatus according to any one of appendixes 1 to 12, wherein the image capturing apparatus and the lateral mirror are installed in such a manner that a distance from the lateral mirror to the reference point is smaller than a distance from the image capturing apparatus to the reference point.

### Appendix 14

The information processing apparatus according to any one of appendixes 1 to 13, wherein the one or more image capturing apparatuses include a first image capturing apparatus that is focused on the vicinity of the reference point and a second image capturing apparatus that is focused on a vicinity of the lateral mirror, and

the processing circuitry is configured to:

determine the first contour based on a first partial image included in an input image captured by the first image capturing apparatus, and

determine the second contour based on a second partial image included in an input image captured by the second image capturing apparatus.

### Appendix 15

The information processing apparatus according to any one of appendixes 1 to 14, wherein the processing circuitry is configured to:

further acquire an input image captured by a top image capturing apparatus installed at an upper side of the reference point,

determine a third contour of the target viewed from a third viewpoint different from the first viewpoint and the second viewpoint based on a third partial image in which the target is captured, the third partial image being included in the input image captured by the top image capturing apparatus, and

present information based on at least one of the first contour, the second contour or the third contour.

### Appendix 16

The information processing apparatus according to any one of appendixes 1 to 14, wherein the processing circuitry is configured to:

further acquire an input image captured by a top-mirror image capturing apparatus installed below a top mirror installed at an upper side of the reference point,

determine a third contour of the target viewed from a third viewpoint different from the first viewpoint and the second viewpoint based on a third partial image in which a mirror image of the target reflected by the top mirror is captured, the third partial image being included in the input image captured by the top-mirror image capturing apparatus, and

present information based on at least one of the first contour, the second contour or the third contour.

### Appendix 17

An information processing apparatus comprising processing circuitry configured to:

acquire (S110) an input image captured by one or more image capturing apparatuses (30) installed at a forward side or a rearward side of a reference point;

determine (S111) a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a mirror installed at the forward side or the rearward side of the reference point and on an opposite side of the reference point to at least one of the image capturing apparatuses is captured; and

present (S113) information based on at least one of the first contour or the second contour.

### Appendix 18

The information processing apparatus according to appendix 17, wherein the mirror is installed at the rearward side of the reference point, and

the processing circuitry is configured to prompt the target to move so that an entire mirror image of the target is included in the input image.

### Appendix 19

A method for operating a computer comprising processing circuitry, wherein the processing circuitry is configured to:

acquire (S110) an input image captured by one or more image capturing apparatuses (30) installed at a forward side or a rearward side of a reference point;

determine (S111) a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a lateral mirror (40) installed at a side of the reference point is captured; and

present (S113) information based on at least one of the first contour or the second contour.

### Appendix 20

A non-transitory computer-readable program for operating a computer apparatus comprising processing circuitry, wherein the program causes the processing circuitry to perform:

acquiring (S110) an input image captured by one or more image capturing apparatuses (30) installed at a forward side or a rearward side of a reference point;

determining (S111) a first contour of a target located in a vicinity of the reference point viewed from a first viewpoint based on a first partial image of the input image in which the target is captured and determining a second contour of the target viewed from a second viewpoint different from the first viewpoint based on a second partial image of the input image in which a mirror image of the target reflected by a lateral mirror (40) installed at a side of the reference point is captured; and

presenting (S113) information based on at least one of the first contour or the second contour.

Although an embodiment of the present invention has been described above in detail, the scope of the present invention is not limited to the embodiment described above. Various improvements or modifications can be made to the embodiment described above without departing from the spirit of the present invention. The embodiment and variations described above can be combined with each other.

1. An information processing apparatus comprising processing circuitry configured to:
acquire an input image captured by one or more image capturing apparatuses installed at a forward side or a rearward side of a reference point;
determine a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a lateral mirror installed at a side of the reference point is captured; and
present information based on at least one of the first contour or the second contour.

2. The information processing apparatus according to claim 1, wherein the target is a user exercising in the vicinity of the reference point, and
the processing circuitry is configured to:
evaluate a posture of a part of a body of the user based on at least one of the first contour or the second contour, and
present, to the user, information based on an evaluation result of the posture of the part of the body of the user.

3. The information processing apparatus according to claim 2, wherein the processing circuitry is configured to present, to the user, advice concerning the posture of the part of the body of the user.

4. The information processing apparatus according to claim 3, wherein the processing circuitry is configured to present, to the user, audio including the advice concerning the posture of the part of the body of the user.

5. The information processing apparatus according to claim 2, wherein a half mirror is installed in front of the reference point, and
the processing circuitry is configured to display the information on a display installed behind the half mirror with respect to the reference point.

6. The information processing apparatus according to claim 5, wherein at least one of the image capturing apparatuses is installed at the rearward side of the reference point, and
the processing circuitry is configured to display a back image of the user based on the first partial image on the display.

7. The information processing apparatus according to claim 2, wherein the processing circuitry is configured to:
calculate depths of corresponding points in the first partial image and the second partial image based on a positional relationship between the image capturing apparatus and the lateral mirror and orientations of the image capturing apparatus and the lateral mirror, and
evaluate the posture of the part of the body of the user based on the depths.

8. The information processing apparatus according to claim 2, wherein the processing circuitry is configured to evaluate an angle of a contour line of the part of the body of the user based on at least one of the first contour or the second contour.

9. The information processing apparatus according to claim 2, wherein the processing circuitry is configured to:
determine a contour line of the part of the body of the user based on at least one of the first contour or the second contour, and
evaluate the posture of the part of the body based on a comparison between the contour line of the part of the body of the user and a reference contour line.

10. The information processing apparatus according to claim 9, wherein the processing circuitry is configured to:
determine a contour line of a back of the user based on at least one of the first contour or the second contour, and
evaluate a distortion of a pelvis of the user or a bending of the back or a waist of the user based on a comparison between the contour line of the back of the user and a reference contour line.

11. The information processing apparatus according to claim 9, wherein the processing circuitry is configured to:
determine a contour line of a toe of the user based on at least one of the first contour or the second contour, and
evaluate an orientation of the toe of the user based on a comparison between the contour line of the toe of the user and a reference contour line.

12. The information processing apparatus according to claim 2, wherein the processing circuitry is configured to control a posture of a corresponding part of an operation target in a virtual space in response to the evaluation result of the posture of the part of the body of the user.

13. The information processing apparatus according to claim 1, wherein the image capturing apparatus and the lateral mirror are installed in such a manner that a distance from the lateral mirror to the reference point is smaller than a distance from the image capturing apparatus to the reference point.

14. The information processing apparatus according to claim 1, wherein the one or more image capturing apparatuses include a first image capturing apparatus that is focused on the vicinity of the reference point and a second image capturing apparatus that is focused on a vicinity of the lateral mirror, and
the processing circuitry is configured to:
determine the first contour based on a first partial image included in an input image captured by the first image capturing apparatus, and
determine the second contour based on a second partial image included in an input image captured by the second image capturing apparatus.

15. The information processing apparatus according to claim 1, wherein the processing circuitry is configured to:
further acquire an input image captured by a top image capturing apparatus installed upper side of the reference point,
determine a third contour of the target viewed from a third viewpoint different from the first viewpoint and the second viewpoint based on a third partial image in which the target is captured, the third partial image being included in the input image captured by the top image capturing apparatus, and
present information based on at least one of the first contour, the second contour, or the third contour.

16. The information processing apparatus according to claim 1, wherein the processing circuitry is configured to:
further acquire an input image captured by a top-mirror image capturing apparatus installed below a top mirror installed at an upper side of the reference point,
determine a third contour of the target viewed from a third viewpoint different from the first viewpoint and the second viewpoint based on a third partial image in which a mirror image of the target reflected by the top mirror is captured, the third partial image being included in the input image captured by the top-mirror image capturing apparatus, and
present information based on at least one of the first contour, the second contour, or the third contour.

17. An information processing apparatus comprising processing circuitry configured to:
acquire an input image captured by one or more image capturing apparatuses installed at a forward side or a rearward side of a reference point;
determine a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a mirror installed at the forward side or the rearward side of the reference point and on an opposite side of the reference point to at least one of the image capturing apparatuses is captured; and
present information based on at least one of the first contour or the second contour.

18. The information processing apparatus according to claim 17, wherein the mirror is installed at the rearward side of the reference point, and
the processing circuitry is configured to prompt the target to move so that an entire mirror image of the target is included in the input image.

19. A method for operating a computer comprising processing circuitry, wherein the processing circuitry is configured to:
acquire an input image captured by one or more image capturing apparatuses installed at a forward side or a rearward side of a reference point;
determine a first contour of a target located in a vicinity of the reference point viewed from a first viewpoint based on a first partial image of the input image in which the target is captured and determine a second contour of the target viewed from a second viewpoint different from the first viewpoint based on a second partial image of the input image in which a mirror image of the target reflected by a lateral mirror installed at a side of the reference point is captured; and
resent information based on at least one of the first contour or the second contour.

20. A non-transitory computer-readable program for operating a computer apparatus comprising processing circuitry, wherein the program causes the processing circuitry to perform:
acquiring an input image captured by one or more image capturing apparatuses installed at a forward side or a rearward side of a reference point;
determining a first contour of a target located in a vicinity of the reference point viewed from a first viewpoint based on a first partial image of the input image in which the target is captured and determining a second contour of the target viewed from a second viewpoint different from the first viewpoint based on a second partial image of the input image in which a mirror image of the target reflected by a lateral mirror installed at a side of the reference point is captured; and
presenting information based on at least one of the first contour or the second contour.
